(19) United States
(12) Patent Application Publication
(10) Pub. No.: US 2005/0273159 A1
(43) Pub. Date: Dec. 8, 2005

(54) Monocusp valve construction and defect closure device for deep vein regurgitation

(76) Inventor: John C. Opie, Scottsdale, AZ (US)

Correspondence Address: SQUIRE, SANDERS & DEMPSEY L.L.P.;Two Renaissance Square, Suite 2700, 40 North Central Avenue, Phoenix, AZ, 85004-4498, US

(21) Appl. No.: 11/042,315

(22) Filed: Jan. 24, 2005

Related U.S. Application Data
(60) Provisional application No. 60/538,255, filed on Jan. 22, 2004.
(60) Provisional application No. 60/607,459, filed on Sep. 3, 2004.

(51) Int. Cl. A61F 2/06

(52) U.S. Cl. 623/1.24; 128/898; 606/213

(57) ABSTRACT

A vein valve, preferably for the common femoral vein, is described. The valve may be of a monocusp or bicuspid construction. The valve is constructed from the patient's own vein wall tissue, in which case it is necessary to repair the opening made in the vein wall with a precisely shaped, minimally thrombogenic vein wall patch, which may be, and is preferably, synthetic. The synthetic patch may be both heparin and antibiotic bonded and thus confer special advantages.

### BACKGROUND OF THE INVENTION

Common femoral vein regurgitation is a common illness affecting millions of patients throughout the world. It can result in heavy swollen legs that are permanently uncomfortable to the patient. Not infrequently, chronic uncorrected deep venous regurgitation results in “woody” fat necrosis of calf subcutaneous tissues. As the condition worsens extensive hemosiderin (brown iron pigment) staining of the lower-third of the shin and calfskin may occur. Further advancement may lead to ankle ulceration that is reluctant to heal, which can be followed by infection and chronic pain. In very advanced circumstances, perhaps as a result of post phlebitic leg syndrome, some legs have required amputation to prevent gangrene.

Several therapeutic interventions have been attempted to cure this condition, and include elastic support hose of either low or high compression. Support hose does not reverse the essential problem, however. Alternatively, some surgeons have tried implanting prosthetic valves, which can be placed in the common femoral vein percutaneously via the jugular vein. This technology has not been widely adopted and has problems with regard to thrombogenicity of the artificial valve. Other therapeutic interventions have included implantation of a competent homograft, femoral vein valve from a cadaver. These have, however, a significant propensity to acutely thrombose and tend to require long-term anticoagulation therapy. There is also a potential risk of hepatitis and HIV being transplanted with donor tissue, and long-term follow up demonstrates intermediate term 2-3 year valve failure with recurrent problems.

Previous studies have shown that approximately 21% of patients with deep venous regurgitation have no valves and another 15-20% have valves that are incompetent. A living, autogenous, monocusp or possible bicuspid valve created from the patient's own vein wall tissues with a synthetic vein closure patch(s) could permanently eliminate chronic venous congestion and would prevent many or all of the complications associated with chronic deep venous regurgitation.

If the patient has valves that are incompetent it is possible to advance the leading valve leaflet edges up the proximal vein wall and thus regain competency of the valve. It should be noted that the valves are extremely thin and if inadvertently damaged at surgery failure to correct venous regurgitation, the surgery will not be successful. Up to now there has been no effective means for successfully preventing, on a long-term basis, the regurgitation in the femoral vein when a patient's own venous valves fail and the valves cannot be repaired. Having the procedure described herein as a back up or alternative to such surgery is thus very useful.

### SUMMARY OF THE INVENTION

In accordance with the present invention, a nonthrombogenic, autogenous, largely or completely competent, common femoral vein valve and vein patch are described. Essentially, the valve is created from the wall of the patient's own femoral vein. Two vertical incisions are made in the vein and a horizontal incision is then made to connect the vertical incisions. Thus, a flap of tissue is formed. The flap is pushed inward so that it is positioned in the vein and is retained in place as a one-way valve, preferably by suturing. The resulting hole in the femoral vein (or any vein in which the invention is used) is patched using a specifically-sized synthetic patch.

A principle object of the present invention is to provide, for the first time, a simple surgical procedure to create a living, autogenous, vein wall monocusp valve (or possibly opposing bicusp valves) that will be able to lessen or eliminate common femoral vein regurgitation and as a result will prevent on a long-term basis, the advanced complications of uncorrected deep vein regurgitation.

One benefit of the present invention is that a valve of living autogenous patient tissue will not be rejected by the patient's body defense mechanisms as is foreign tissue.

Another benefit of the present invention is that the valve is lined by living interior wall endothelium facing the blood stream. As such it is nonthrombogenic and will not require long-term anticoagulation therapy.

Another benefit of the present invention is that the valve will be adequately managed with simple antiplatelet medication for the prevention of deep vein thromboses.

It is to be understood that both the foregoing general description and the following detailed description are exemplary and explanatory only and are not restrictive of the invention, as ultimately claimed.

The accompanying drawings, which are incorporated in and constitute a part of this specification, illustrate one or more preferred embodiments of the invention and together with the description, serve to explain the principles of the invention.

### BRIEF DESCRIPTION OF THE DRAWINGS

FIG. 1 is a plan, cross-sectional view of a vein showing the locations of the preferred incisions to create a valve according to the invention.

FIG. 2 is a side view of the vein of FIG. 1 showing the valve depressed into the lumen of the vein.

FIG. 3 is a plan view of the two proximal sutures loosely placed to allow monocusp movement. The anterior vein wall is tented.

FIG. 4 is a side view of the vein with the valve therein and having with the loosely-placed sutures applied. The sutures allow the valve to flex open and closed. The sutures also prevent the valve from prolapsing. Three valve positions are shown.

FIG. 5 is a plan view showing two distal “cocking” sutures

FIG. 6 is a side, cross-sectional view of the valve in the fully open position and the closing synthetic patch repairing the anterior vein wall defect.

FIG. 7 is a side, cross-sectional view of vein with a closed valve.

FIG. 8 is a side view showing the synthetic vein wall patch sewn into place and covering the hole created to form the valve.

FIG. 9 is a cross-sectional, top view of the vein from above looking down onto the valve.

FIG. 10 is a cross-sectional, top view of the vein looking down on the closed valve in simulated regurgitation. The loose sutures allow the monocusp to closely approximate the back wall of the vein but do not permit the monocusp to prolapse and permit wide-open reflux to occur.

FIG. 11 is a plan view of a synthetic patch according to the invention.

FIGS. 12-18 are other images of the present invention.

### DETAILED DESCRIPTION OF THE DRAWINGS

Reference will now be made in detail to the preferred exemplary embodiments of the invention, examples of which are illustrated in the accompanying drawings.

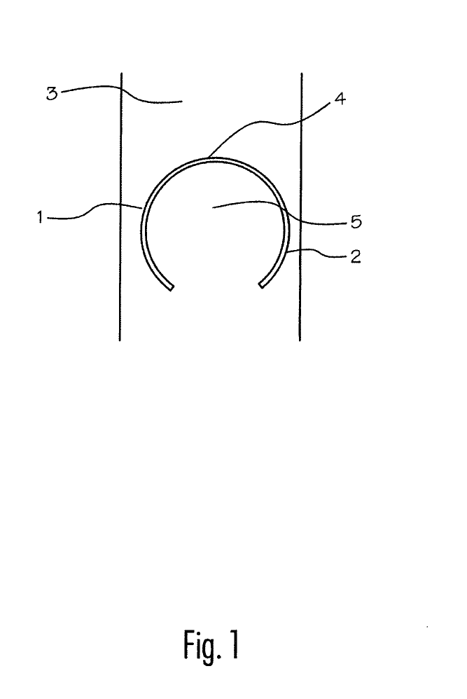

FIG. 1 shows two vertical medial incisions 1 and a lateral incision 2 to be made along the anterior wall of a vein (which is preferably a common femoral vein) 3 to develop a flap (or monocusp valve) 5. As each individual's vein will vary somewhat in diameter the vertical cuts need to be as long as the diameter of the vein with preferably about a 1% to 20% and most preferably about a 10% margin of excess to ensure that flap is long enough to function well as a valve. A slightly convex incision may be useful in that the valve will more closely approximate the shape of the lateral wall of the round distended vein when full of blood. (Note that manipulation of the vein results in vein spasm and it is useful to irrigate the external surface of the common femoral vein with lidocaine or Papavarin to prevent autonomic spasm from occurring.) Once the two vertical cuts are made they are joined proximally by the horizontal incision 4.

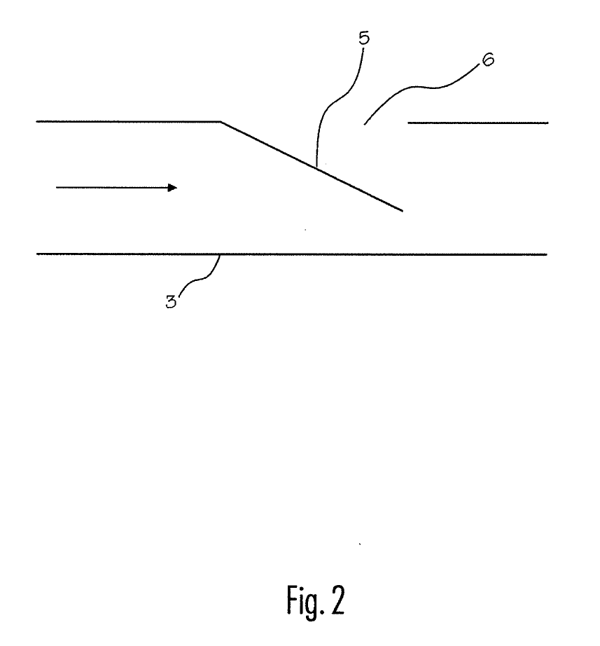

FIG. 2 shows the valve (shown as a monocusp flap) 5 depressed into the lumen of the vein. So long as the monocusp 5 is not opposed to the opposite wall the flow is antegrade in the lumen. The vein has no reparative patch in defect (or hole) 6 at this time.

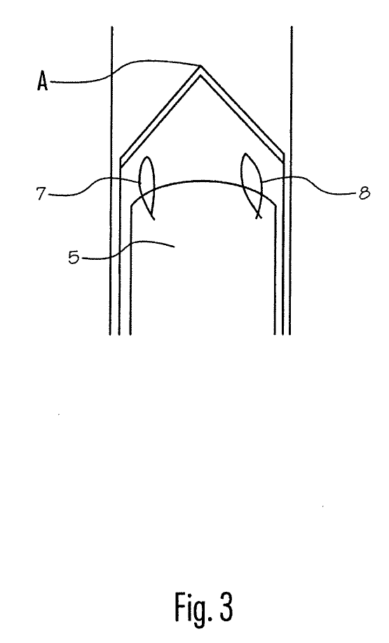

FIG. 3 is a plan view of the two 6-0 prolene or similar sutures 7 and 8 placed at the corners of the valve 5 and sutured to the back wall of the common femoral vein 3. A is a traction suture (and is preferred, but not required) 7 and 8 to better visualize the valve sutures 7 and 8. It is removed once sutures 7 and 8 are placed.

The two prolene sutures preferably pass through the corners of the flap and stretch loosely across the lumen of the vein and are sutured to the postero-lateral and postero-medial wall of the intact vein posterior wall. The sutures are preferably loose enough to permit the monocusp valve to open up to 90-97 percent allowing the monocusp to approximate the anterior wall of the vein with antegrade venous flow.

Further, the two prolene sutures are loose enough to permit the monocusp valve to oppose against the posterior wall and not prolapse by virtue of the two proximal corner sutures and thus temporarily closing off most of the lumen of the common femoral vein preventing most of the regurgitant back flow.

Two additional distal prolene sutures may be placed at the distal base of the monocusp valve 5. In that case, the sutures are placed at both sides of the base of valve 5. These sutures pass horizontally through the external surface of the monocusp valve 5 slightly medial to the distal termination of the vertical antero-lateral and antero-medial cuts. The suture is then brought inside the vein and passed from inside to outside at the distal termination of the antero-medial and antero-lateral vein walls adjacent to the base of the monocusp 5. These two sutures are then tied outside the vein. This pushes the base of the monocusp valve 5 inside the vein at all times and thus “cocks” the monocusp 5. This ensures the valve will remain “cocked” and will close any time there is any tendency for venous back flow. It will also slightly crimp the vein. The sutures at each side of the base of the monocusp 5 should be close to the edge of monocusp 5 so as to minimize this crimping effect.

The monocusp valve 5 preferably moves to an almost fully open position with antegrade flow and a monocusp flap of vein wall that closes against the opposing wall and fully closes with, any potential back flow due to the fact that it is “cocked” at all times. Further, valve 5 may permit some lateral back flow along its sides where such back flow will not completely contact the medial and lateral wall of the vein.

In the case of a synthetic patch used with the invention, the patch needs to be approximately as wide as the common femoral vein (roughly the width of an adult human thumb at the first interphalangeal joint), and slightly longer than the width of the vein. Since the valve needs to come into contact with posterior wall of the vein (to limit pressurized venous reflux) the patch needs to be slightly longer than the length of the flap or valve, which will be approximately 1.1 to 2.0, and most preferably 1.5, times the vein diameter.

It is possible to halve the lengths of the monocusp and create similar cuts on an opposing side of the vein so as to create two flaps that form a singe valve. This will result in a bicuspid valve arrangement. Such a series of mirrored cuts on opposing walls will require two vein defect (or hole) closure patches—one on either side of the vein. The vein wall is delicate tissue and thus easily damaged. A bicuspid valve requires very meticulous surgery and may offer no specific advantage and is not preferred although it is possible.

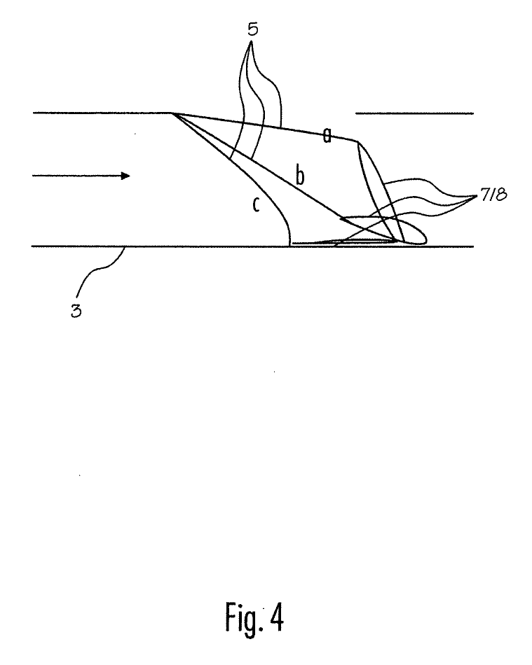

FIG. 4 shows some various positions of the monocusp valve 5, namely a, b and c. “a” is when the valve is fully open and “c” is when the valve is fully closed. These position changes can only occur if the two sutures 7 and 8 are placed inside the vein in a loose manner. The length of the loop of suture describes the arc of movement of the valve 5. Sutures 7 and 8 should be most approximately the diameter of the vein, and preferably between 60%-140% the diameter of the vein.

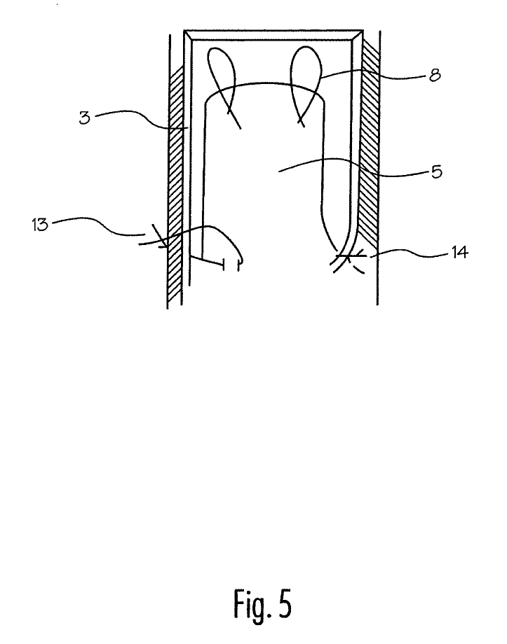

FIG. 5 is a plan view of the placement of the two “cocking” sutures. In this view, the medial suture 13 has been placed but untied. The lateral suture has been tied 14, showing how the monocusp is “cocked” and positioned slightly inside the vein wall. By tying the suture 13 the monocusp valve 5 will be placed under the vein wall 3 and the valve 5 is thus “cocked” at all times.

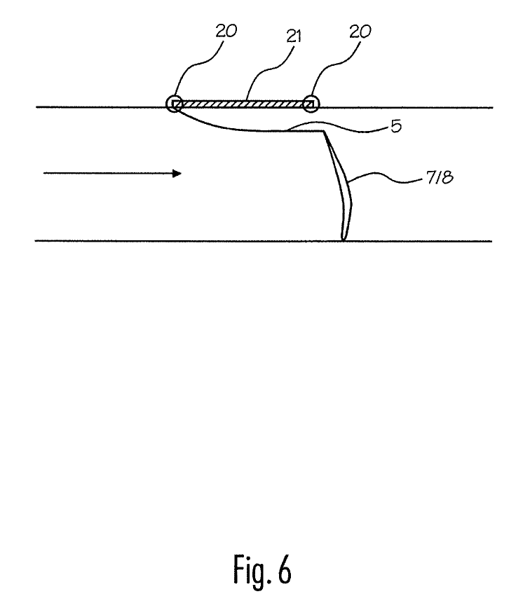

FIG. 6 shows the vein in a lateral view and shows the monocusp 5 open to permit unobstructed antegrade flow. Valve 5 is prevented from reaching the fully open position by the tethering effect of the loose sutures 7 and 8. The lateral wall defect is now repaired with a synthetic vein wall patch 21, preferably sewn into place with a running 6-0 prolene suture 20.

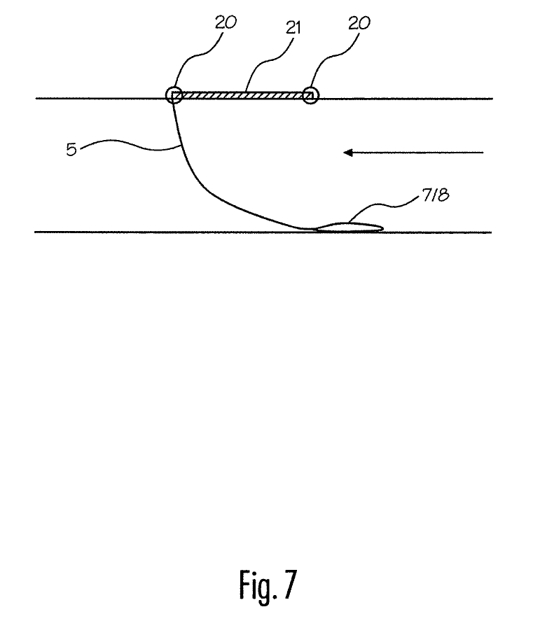

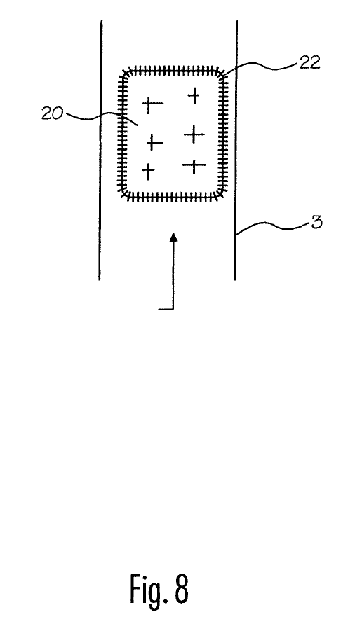

FIG. 7 shows the monocusp in the fully closed position and limiting or eliminating regurgitation. The two sutures 7 and 8 prevent the monocusp from prolapsing and permitting uncontrolled reflux. FIG. 8 shows the synthetic vein patch 20 used to close the anterior vein wall defect. The patch is also preferably sewn into place with a running 6-0 prolene suture 22 in such a fashion so as to not disturb the tubular structure of the common femoral vein 3.

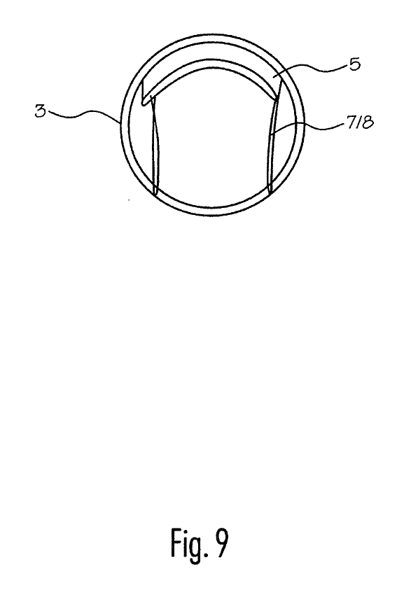

FIG. 9 is a view of the vein from above down showing the “cocked” valve in the fully open position and restrained by the two sutures 7 and 8.

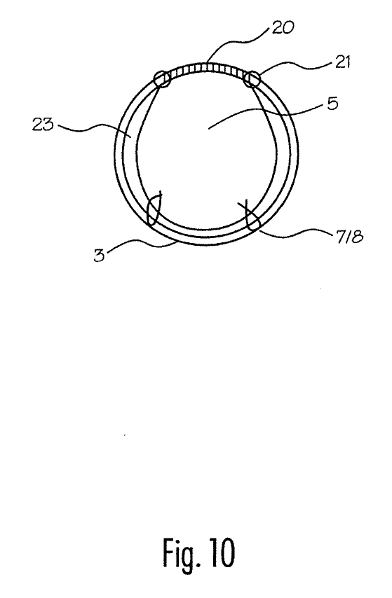

FIG. 10 is a similar view as FIG. 9 but in this instance the monocusp 5 is in the fully closed position. The sutures are now resisting any tendency for prolapse. The patch 20 is now sutured in place, preferably with a running suture 21. It should be obvious that some regurgitation will occur through space 23. However, the volume of reflux via this small space is likely to be quite limited.

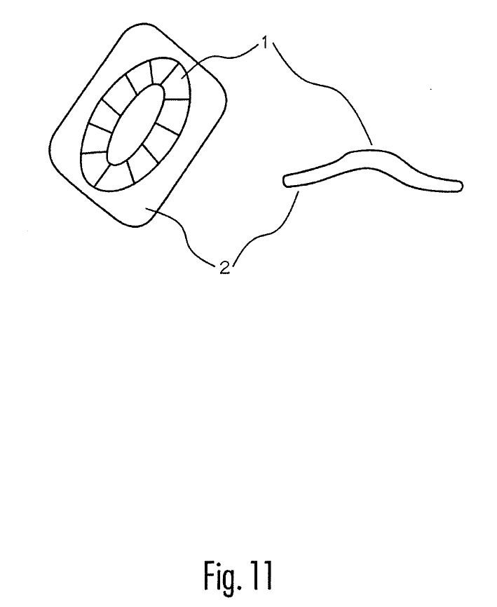

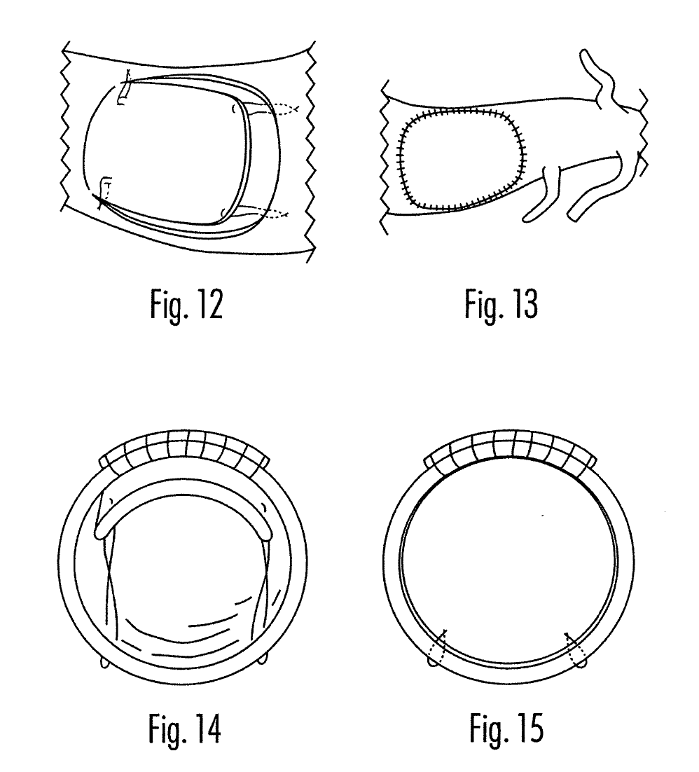

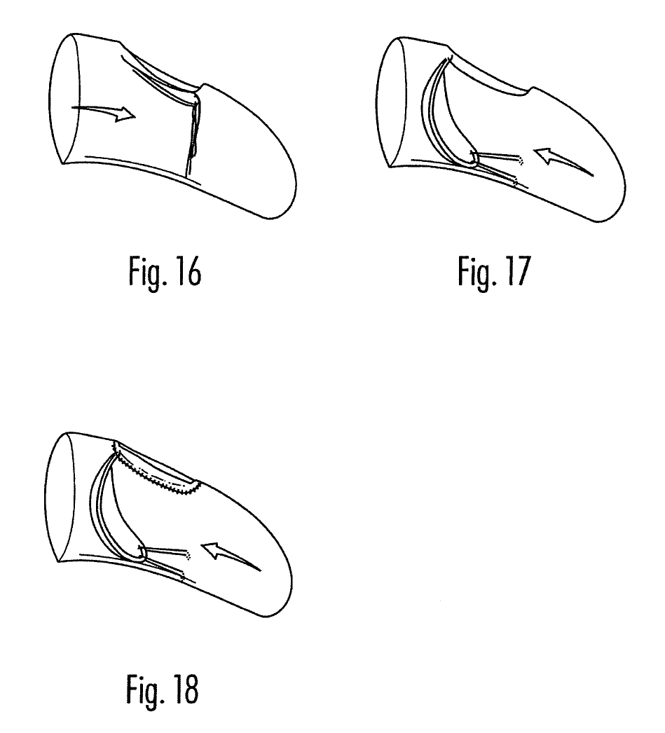

FIG. 11 is a plan view of an optional bubble patch. The flange for sewing into the vein 2 is visible and the bubble itself 1 is visible. The bubble would help to separate the monocusp 5 and the patch.

The bubble is preferably in the center of the patch, is smaller than the width and length of the synthetic patch and thus leaves a flange outside the bubble for suturing. The bubble extends out from the center of the patch for a short distance when the patch is distended with venous blood. The distended bubble is designed to keep the open, tethered, monocusp 5 physically separated from the inner surface of the synthetic patch as much as possible, so as to limit the possibility of the patch and the inner surface of the patch from coming into contact with each other and possibly adhering thus frustrating the monocusp 5 and essentially gluing it in the open position.

Any synthetic patch according to the invention is made of an appropriate, highly compliant material, such as very thin e-PTFE. The patch preferably does not distort the vein when the vein vascular clamps are released after the procedure is complete and venous flow is restored.

Further, a vein patch according to the invention may be treated, such as being surface bonded, with a surface-passive anticoagulant to limit venous thrombosis. (Thrombosis of blood on surfaces is sensitive to several features including total surface area, crystallinity, hydrophobicity /hydroplilicity, outermost structure and surface chemistry. Chemicals bonded to the patch could utilize heparin, stabilized albumin nanoparticles Benzylkonium, Hyaluronan, Trillium or others.)

Additionally, if suitable natural tissue is available, such as the proximal portion of the patient's own long saphenous vein (LSV), this could be opened, shaped and also be utilized for the vein closure patch. Many patients however, have previously undergone long saphenous vein stripping procedures and the LSV tissue is not available. Under these circumstances a synthetic vein patch will be necessary. More than one monocusp valve is possible may be formed, but likely not more than one required to control reflux long term.

The creation of the monocusp is a routine surgical procedure. The “cocking” sutures are obvious and necessary to ensure the monocusp will tend to oppose the contralateral posterior vein wall when regurgitation occurs as are the monocusp limiting proximal sutures, which are designed to prevent complete monocusp prolapse when the venous blood regurgitation pressure rises.

The CFV patch should be approximately 12 mm wide with a very slight lateral and medial convexity and a proximal and distal shape that is also slightly convex to conform to the shape of the CFV. The vein patch needs to be (preferably) approximately 15 mm long or one and a half times the diameter of the CFV. Due to the very thin wall of the CFV the patch is best made of very thin PTFE. It could be made of thin Dacron or Teflon as well. During insertion of the patch it might be useful to soak the patch in an antibiotic solution.

In summary, an improved, nonthrombogenic, endothelialzed, living tissue, surgical procedure for the control of common or deep leg vein incompetence is described.

Thirdly, nonviable valvular structures have no capacity to produce endothelial prostacyclin, which is well understood to protect against local blood vessel wall thrombosis. Because the monocusp is pedicled on a distal uncut bridge, it is a viable valve and as such the endothelized surface has full prostacyclin production capabilities.

The vascular patch may be somewhat thrombogenic however it only occupies about 40% of the anterior vein wall. The opposing wall is the external surface of the monocusp, which was the outside of the vein initially. The monocusp outer wall will be constantly moving opening for antegrade flow and closing to prevent reflux. That regular and intermitted movement will help prevent the inner patch wall and the outer surface of the monocusp from clotting. After a short time it is known that most of these patches develop some degree of endotholization. It is expected that the outer or superior surface of the monocusp will be entirely endothelialized within a short time since it is a living structure.

At all times when operating on the common femoral vein long term thrombogenicity is always an issue due to the fact that if a clot were to develop and possibly embolize this could result in a serious pulmonary embolism. Due to the fact that natural living autologous tissue is being used to create the viable monocusp valve it is recommended that either ASA or Plavix only be used postoperatively. It is not necessary to use anticoagulants such as coumadin.

Coumadin is not necessary and can be problematical with continuing bleeding from the venous suture lines and lead to worrisome wound hematomas. Most patients can be discharged three to five day after surgery once the drain has been removed.

The level of monocusp competency can be assessed easily with postoperative Venous Duplex Doppler examinations with Valsalver maneuvers and or local compression assessments. In all cases where venous reconstruction surgery is performed, the venous pressure is typically <8 mm Hg. It is not similar to arterial surgery where much higher pressures operate. Therefore, due to the lower pressures, it might be possible for a wound hematoma to develop after surgery since all of these valvular reconstructions are performed while the patient is systemically anticoagulated with heparin. It is necessary to control every branch of the common femoral vein (of which there are many) and as well it is necessary to have a proximal and distal occlusion clamp on the femoral vein and profunda vein. Furious hemorrhage will occur if this is not done. Significant venous blood loss lowers cardiac preload and is poorly tolerated by anesthetized patients and very quickly leads to a pronounced drop in cardiac output. Careful attention to technique can eliminate this risk.

While this invention has been described in terms of its preferred embodiments and various modifications, those skilled in the art will appreciate that other modifications can be made without departing form the spirit and scope of this invention. The invention is thus not limited to this embodiments disclosed herein, but is instead set forth in the following claims and legal equivalents thereof.

It is wise to cover these prosthetic vein patch implants with parenteral antistaphlococcal antibiotics. Venous prosthetic patches if they became infected would present a substantial problem and could result in centrally progressive septicemia. An infected synthetic vein patch could possibly be explanted but another patch would be necessary and the new implant would have to be in an infected situation. It might also become infected.

Side-biting vascular clamps are undesirable due to the distortion they create in the vein. Such distortion makes it difficult to assess where to make the incisions in the anterior common femoral vein wall and how to make the monocusp. The back wall is tented with side-biting clamps and the placement of the posterior sutures is also confusing.

Due to the risk of a wound hematoma it is wise to use a wound drain in every case to limit the possibility of a wound hematoma. Such a wound hematoma could occlude the low-pressured femoral vein and lead to a deep vein thrombosis and increase the possibility of embolic events.

Because of the multitude of common femoral vein branches it is valuable to make the incision in the antero-medial thigh slightly longer than one would make for a femoral artery exposure. The reason for this is to provide an undistorted 2-3 inch segment of external iliac-common femoral-superficial femoral vein and then create the monocusp in the proximal common femoral vein segment with the vein now undistorted.

1. A method of vein valve construction of a vein having a wall and a lumen, wherein the method includes the steps of:
a) making a first vertical incision in the vein wall;
b) making a second vertical incision in the vein wall;
c) making a horizontal incision joining the two vertical incisions to create a flap of vein wall; and
d) depressing the flap of vein wall into the lumen of the vein wherein the flap functions as a valve.

2. The method of claim 1 wherein the vein is the common femoral vein.

3. The method of claim 2 wherein the first vertical incision is in the antero-medial aspect of the vein wall.

4. The method of claim 2 wherein the second vertical incision is in the antero-medial aspect of the vein wall.

5. A method of claim 1 that further includes the step of preventing prolapse of the flap.

6. The method of claim 5 wherein the flap has corners attached to the vein wall and prolapse is prevented by suturing the corners of the flap.

7. The method of claim 1 wherein a hole is left in the vein where the flap was formed and the hole is patched.

8. The method of claim 7 wherein the patch is synthetic.

9. The method of claim 7 wherein the patch is preformed to be substantially the same size as the hole.

10. The method of claim 8 wherein the patch is pretreated with one or more antibiotics.

11. The method of claim 8 wherein the patch is pretreated with one or more anticoagulants.

12. The method of claim 8 wherein the patch is pretreated with one or more anticoagulants and one or more antibiotics.

13. The method of claim 6 wherein the sutures are prolene sutures.

14. The method of claim 13 wherein the valve can open up to 90-97%.

15. A synthetic patch for a vein wall, wherein the patch is precut to a size to be utilized to repair a hole created by a procedure in which part of the vein wall was cut to use as a valve in the vein.

16. The patch of claim 15 that is pretreated with one or more anticoagulants.

17. The patch of claim 15 that is pretreated with one or more antibiotics.

18. The patch of claim 15 that is pretreated with one or more anticoagulants and one or more antibiotics.

19. The patch of claim 15 that comprises ePTFE.

20. The method of claim 8 wherein the patch comprises ePTFE.

21. The patch of claim 15 that includes a bubble.

22. The patch of claim 21 wherein the bubble is centrally positioned on the patch.
